(19) United States
(12) Patent Application Publication
(10) Pub. No.: US 2021/0042294 A1
(43) Pub. Date: Feb. 11, 2021

(54) BLOCKCHAIN-BASED CONSENT MANAGEMENT SYSTEM AND METHOD

(71) Applicant: DLT Labs, Inc., Toronto (CA)

(72) Inventor: Neeraj Srivastava, Toronto (CA)

(73) Assignee: DLT Labs, Inc., Toronto, ON (CA)

(21) Appl. No.: 16/969,126

(22) PCT Filed: Feb. 12, 2019
(86) PCT No.: PCT/CA2019/050177

§ 371 Date: Aug. 11, 2020

Related U.S. Application Data
(60) Provisional application No. 62/629,412, filed on Feb. 12, 2018.

(51) Int. Cl. G06F 16/23; G06F 16/958; H04L 29/06

(52) U.S. Cl. CPC G06F 16/2379; H04L 63/0823; G06F 16/958

(57) ABSTRACT

A blockchain-based consent management system includes a webserver subsystem configured to receive and handle authorized web user requests for access to and/or transactions corresponding to consent data for a blockchain, the webserver subsystem comprising a blockchain subsystem interface; and a blockchain subsystem defining a channel having at least two organizations, corresponding chaincode and an endorsement policy, each of the at least two organizations having at least one peer, each of the at least one peer maintaining a blockchain copy, the blockchain subsystem comprising an orderer in communication with the blockchain subsystem interface.

### CROSS REFERENCE TO RELATED APPLICATION

This application claims priority to U.S. Provisional Patent Application Ser. No. 62/629,412 filed on Feb. 12, 2018, the contents of which are incorporated herein by reference in their entirety.

### FIELD OF THE INVENTION

The following relates generally to computer-implemented healthcare and other patient and stakeholder consent management systems, and more particularly to a blockchain-based consent management system and method.

### BACKGROUND OF THE INVENTION

Proper and efficient obtaining, managing, maintaining and sharing of consents for healthcare provision systems is challenging. This is particularly because patients' personal or healthcare related information, including consents, is considered highly sensitive such that it requires careful and secure handling. Furthermore, the number of stakeholders in healthcare systems—patients, regulators, doctors, hospitals, clinics, emergency personnel, pharmaceutical companies conducting clinical trials, regulators, and the like—each having respective methods of collecting and managing consents, is large and varied. Furthermore, there are various types of consents, each having respective considerations. For example, consents can include: consents to treat a patient, consent to protect information for a patient, patient rights, marketing consents, auto-dial consents, customizable consents, informed consents, and consents to share information. Other forms of consents are possible and may be required as healthcare regulations, treatments and systems evolve. Consents may differ across jurisdictions, and may differ depending on the nature or age of the patient and his or her capacity to grant them.

Various systems have been proposed for centralizing the management of consents for patients and other healthcare system stakeholders. More recently, systems have been proposed that leverage blockchain for immutable and long-term storage of data regarding consents and for controlling access to the immutable consent records for patients. For example, United States Patent Application Publication No. 2018/0082023 to Curbera et al. discloses a secure distributed patient consent and information management system and method for enabling one health provider to directly request patient information from another health provider using a patient record locator provided by a master patient record index.

While systems and methods have been proposed, improvements are desirable.

### SUMMARY OF THE INVENTION

In accordance with an aspect, there is provided a blockchain-based consent management system comprising a webserver subsystem configured to receive and handle authorized web user requests for access to and/or transactions corresponding to consent data for a blockchain, the webserver subsystem comprising a blockchain subsystem interface; and a blockchain subsystem defining a channel having at least two organizations, corresponding chaincode and an endorsement policy, each of the at least two organizations having at least one peer, each of the at least one peer maintaining a blockchain copy, the blockchain subsystem comprising an orderer in communication with the blockchain subsystem interface.

In an embodiment, the blockchain subsystem and the webserver subsystem communicate such that authorized requests for transactions for the blockchain cause the webserver subsystem to generate transaction proposals to be routed via the blockchain subsystem interface to the channel peers of the blockchain subsystem for individual endorsement, to receive endorsement responses from the peers, and to, in the event the endorsement responses collectively satisfy the endorsement policy, transmit the endorsed transactions to the orderer for inclusion in an additional block of the blockchain, wherein the orderer is configured to cause the additional block to be stored in each of the peers' blockchain copy.

In an embodiment, the webserver subsystem is in communication with a certificate authority for the system.

In an embodiment, the webserver subsystem comprises a first auto-scaling group in communication with a second auto-scaling group via a request queue, wherein the first auto-scaling group lodges requests corresponding to user requests in the request queue for consumption by the second auto-scaling group.

In an embodiment, the first auto-scaling group comprises one or more Node JS webservers having user interface and application middleware components.

In an embodiment, the second auto-scaling group comprises one or more Node JS webservers having instances of a hyperledger fabric interface.

In an embodiment, the webserver subsystem further comprises load balancing components for routing the user requests to webserver instances in the first auto-scaling group.

Embodiments disclosed herein provide various advantages. For example, a blockchain-based system provides benefits of security, confidentiality and auditability in order to protect consent information. The consent information stored as disclosed herein in a blockchain is immutable and extremely difficult to penetrate without proper authorization, i.e., to hack.

Embodiments employing the web-based application provide powerful controls over access and additions to blockchain data without imposing a significant limitation on how authorized users can interact with the system and its data, and is suitable for enabling deployment of other application features that may not directly relate to the blockchain data. Embodiments of the system disclosed herein are also easily scaled to incorporate additional entities such as organizations and peers, to deploy new or more sophisticated business logic via new or modified chaincodes, and to adjust and deploy access control lists. The flexibility of the system permits the network to grow, become more sophisticated, to adapt to regulatory and other changes, and generally to become more valuable to its users without undue disruptions as it does.

Embodiments disclosed herein address potential network traffic bottlenecks by providing auto-scaling groups and/or load balancing and/or use of cloud services to automatically expand and contract the system or to provide better geographic availability in response to increases and decreases in user-bases following from changes made to organizations, additions or removal of organizations, peers and other modifications to the network.

Various other embodiments and advantages will become apparent from the following description and drawings.

### BRIEF DESCRIPTION OF THE DRAWINGS

Embodiments of the invention will now be described with reference to the appended drawings in which:

FIG. 1 is a schematic diagram showing a blockchain-based consent management system, according to an embodiment;

FIG. 2 is an enlarged schematic diagram showing components of a webserver subsystem of the blockchain-based consent management system of FIG. 1;

FIG. 3 is an enlarged schematic diagram showing components of a blockchain subsystem of the blockchain-based consent management system of FIG. 1; and

FIG. 4 is a schematic diagram showing a hardware architecture of a computing system suitable as a hardware platform for one or more components of the blockchain-based consent management system of FIG. 1, according to an embodiment.

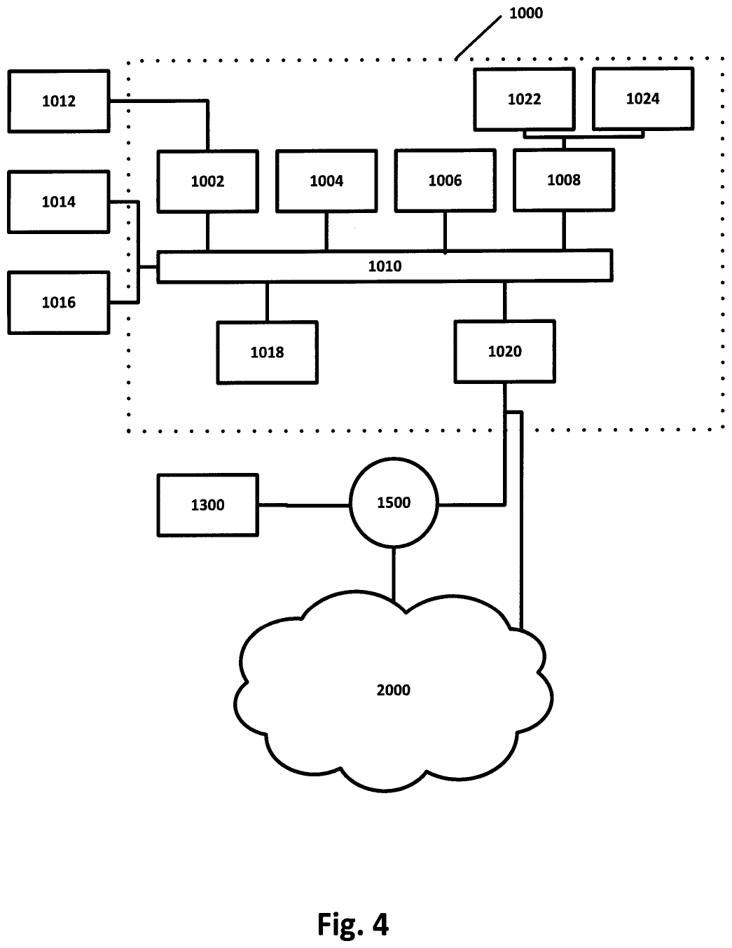

### DETAILED DESCRIPTION

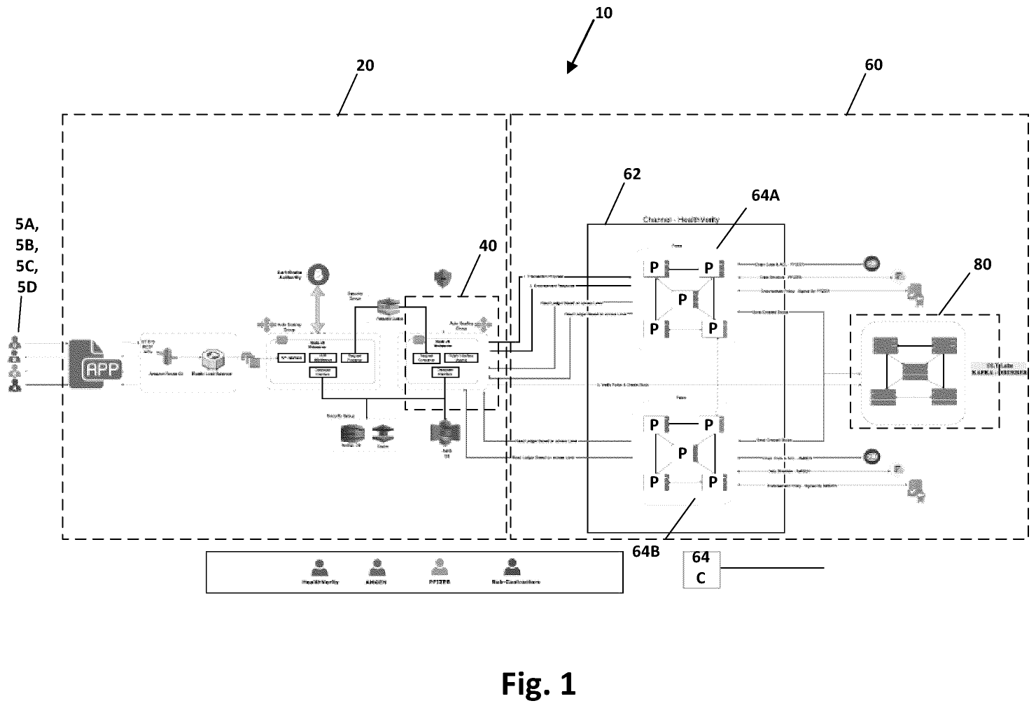

FIG. 1 is a schematic diagram showing a computer-based blockchain-based consent management system 10, according to an embodiment. Blockchain-based consent management system 10 according to this embodiment, is implemented using aspects of the hyperledger fabric framework (see, for example, https://www.hyperledger.org/projects/fabric). In this embodiment, a webserver subsystem 20 is configured to receive and handle authorized web user requests from users 5A, 5B, 5C, 5D and any other network participants for access to and/or transactions corresponding to consent data for a blockchain, as well as for access to other functionality not directly related to the blockchain. Webserver subsystem 20 includes a blockchain subsystem interface 40. Blockchain-based consent management system 10 also includes a blockchain subsystem 60 in communication with the blockchain subsystem interface 40 of webserver subsystem 20. In this embodiment, blockchain subsystem 60 defines a channel 62 having multiple organizations 64A, 64B, 64C etc., corresponding chaincode and an endorsement policy (not shown), each of the at least two organizations 64A, 64B etc. having, in this embodiment, multiple peers P. Each of the multiple peers P of organizations 64A, 64B etc. of channel 62 maintains a blockchain copy (not shown). In this embodiment, at least one peer P for each organization 64A, 64B etc. is designated as an anchor peer, enabling it to communicate with the anchor peer of another organization in the channel 62. In this embodiment, the bottom right peer P of organization 64A is an anchor peer and the top right peer P of organization 64B is an anchor peer. An orderer 80 of blockchain subsystem 60 is in communication with blockchain subsystem interface 40.

In operation, blockchain subsystem 60 and webserver subsystem 20 communicate such that authorized requests for transactions for the blockchain via a web application 22 cause webserver subsystem 20 to generate transaction proposals to be routed via the blockchain subsystem interface 40 to the channel peers P of the blockchain subsystem 60 for individual endorsement, and to receive endorsement responses from the peers P. In the event the endorsement responses collectively satisfy the endorsement policy, transmit the endorsed transactions to the orderer 80 for inclusion in an additional block of the blockchain. Furthermore, orderer 80 is configured to cause the additional block to be stored in each of the blockchain copy maintain by peers P.

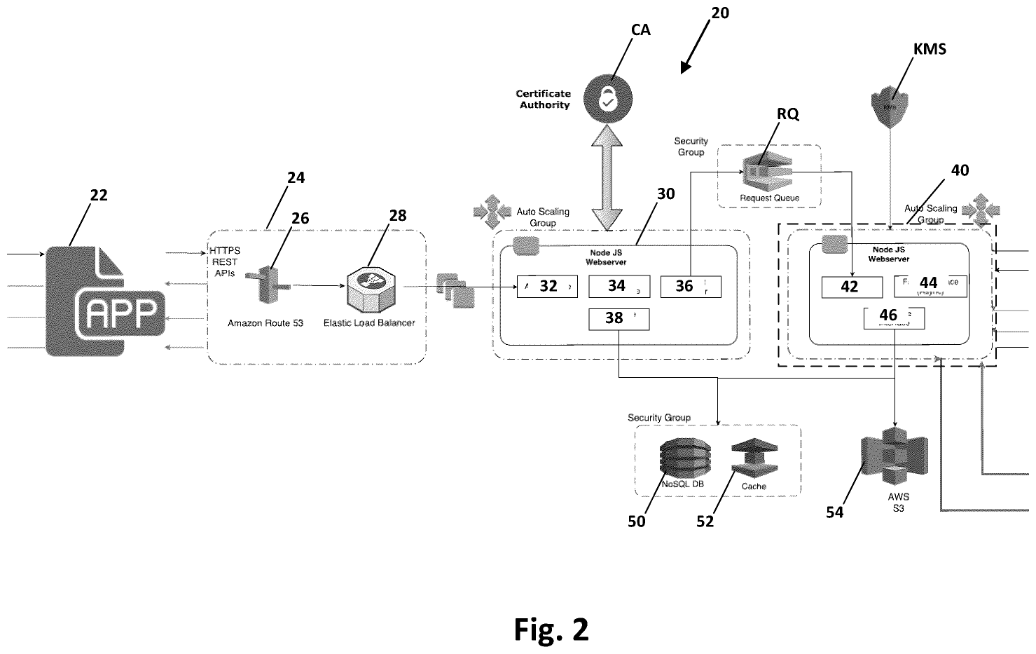

FIG. 2 is an enlarged schematic diagram showing components of webserver subsystem 20 of blockchain-based consent management system 10, according to this embodiment. Web application 22, accessible through individual web browsers run on devices being used by network participants, provides authenticated access to webserver subsystem 20 for, in turn, providing access to the blockchain or for other functions not directly related to the blockchain.

In this embodiment, application 22 is deployed to web browser making requests of webserver subsystem 20 using HTTPs REST APIs (Hypertext Transfer Protocol, Representational State Transfer, Application Programming Interfaces), and is served from one of potentially several instances of a Node JS webserver 30 in an auto-scaling group of webserver subsystem 20. An auto-scaling group is a mechanism for enabling multiple instances of the Node JS webserver 30 to be instantiated or wound down, as required, to efficiently handle rises and falls in incoming traffic loads. A load balancing subsystem 24 for handling and distributing traffic, in this embodiment, includes an Amazon Route 53 DNS service 26 combined with an ELB (Elastic Load Balancer) 28.

The instance of the Node JS webserver 30 from which application 22 is served is representative of all members of its auto-scaling group, in that it provides an API interface 32, Auth Middleware 34, a Database Interface 38 and a Request Publisher 36. API interface 32 provides user interface code executable for deploying the user interface to users' web browsers for interacting with application 22 and interaction with a user for generating read requests, initiating transactions, and other operations. Auth Middleware 34 provides an interface to a Certificate Authority (CA) service. As would be understood, the CA service generates identifiers for each entity and participant in the network, particularly by issuing and maintaining cryptographically validated digital certificates complying with X.509 standard, thereby to authenticate and link identities such as peers P, organizations 64A, 64B, 64C etc., orderer 80, and the like. An MSP (Membership Service Provider) configuration is stored locally at each peer P and at orderer 80. Database Interface 38 provides controlled access to non-blockchain, security-related data maintained by a Security Group in, in this embodiment, a NoSQL DB 50 with caching support 52. The term Security Group is used to refer to Amazon's set of network security policies, as described in, for example, https://blog.learningtree.com/understanding-amazon-ec2-security-groups-and-firewalls.

Database Interface 38 also provides controlled access to a cloud storage service 54, in this embodiment an Amazon Simple Storage Service (Amazon S3), for storing supporting documentation and other data relating to consents being stored on the blockchain. For example, Amazon S3 54 will store deployment- and configuration-related assets, like Docker images of the Node JS webserver 40 that would be used to create new instances of it in its own auto scaling process.

Request Publisher 36 interfaces with a Request Queue RQ that is maintained by the Security Group in order to lodge requests, made in response to user requests provided via application 22.

A second auto-scaling group of Node JS webservers 40 is provided, primarily to serve as the blockchain subsystem interface. Each of Node JS webservers 40 in this second auto-scaling group includes a Request Consumer 42 that interfaces with the Request Queue RQ in order to draw off requests for further handling with respect to the blockchain. Each of Node JS webservers 40 also includes a respective Database Interface 46 for controlled access to non-blockchain, security-related data maintained by a Security Group in the NoSQL DB 50 with caching 52. Database Interface 46 also provides controlled access to the Amazon S3 cloud storage service 54. Node JS webserver 40 also interfaces with a Key Management System, in this embodiment Amazon KMS.

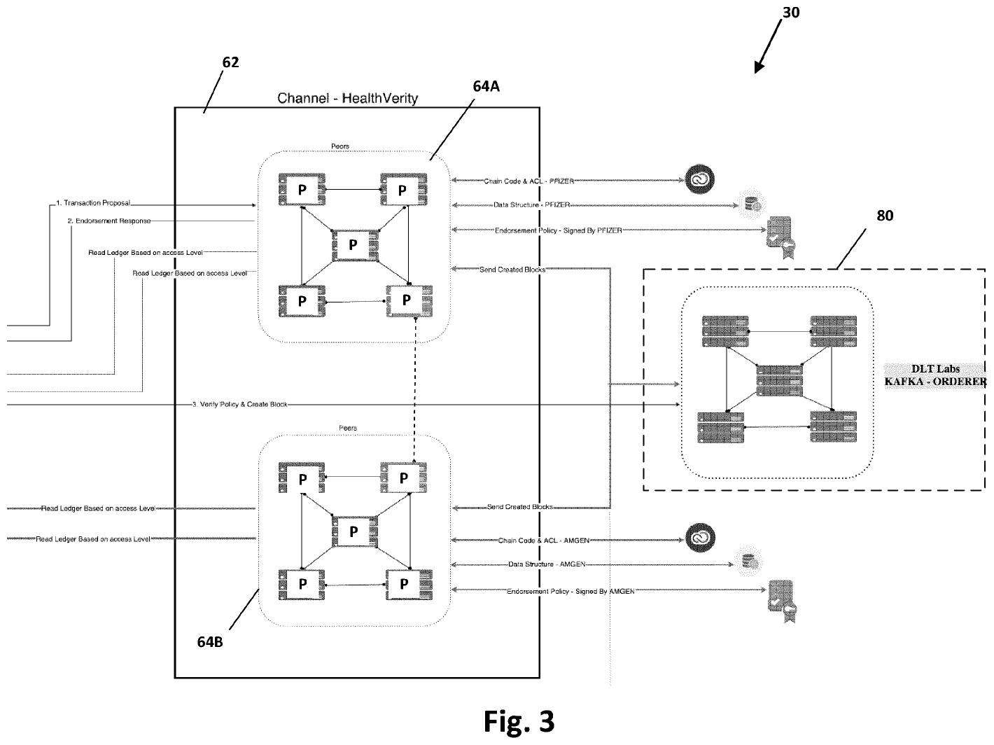

A Fabric Interface 44 enables Node JS webserver 40 to interface with blockchain subsystem 60. FIG. 3 is an enlarged schematic diagram showing components of blockchain subsystem 60. In this embodiment, blockchain subsystem 60 is a hyperledger fabric instance maintaining a channel 62 particularly for handling storage, maintenance and access to consent data for participants in the network.

Channel 62 is a logical structure for managing a respective blockchain and enables the formation of a consortium around private data, such as the particular clients of a health information verification organization established to promote management of consents for and across the clients. In this embodiment, the consortium is shown to include two organizations 64A and 64B. Orderer 80 is, in this embodiment, a distributed Kafka and Zookeeper orderer service.

According to the hyperledger fabric framework, channel 62 has associated with it one or more chaincodes (smart contracts establishing business logic), corresponding endorsement policy(ies) and an access control list (ACL). In this embodiment, ACL implements a consent expiry check to block access to a consent after its respective expiry date. These channel attributes are each stored on each peer P whose organization 64A, 64B etc. has authorized the peer P for inclusion. Each organization in channel 62 may also be a part of another, different channel that maintains a different blockchain and is not affected by or accessible through channel 62. For example, in this example, organization 64A has its own chaincode and endorsement policies for a separate channel, and organization 64B has its own chaincode and endorsement policies for another, separate channel. Organizations 64A and 64B are, along with Other Organizations (64C, for example), also part of channel 62 in order to handle business logic corresponding to consent management with each other and any other organizations that may be added to the channel.

In this embodiment, a particular patient record is an asset in the blockchain of channel 62 and includes at least a portion of the data shown in Table 1, below:

TABLE 1

asset Patient identified by id {

• String id
• String firstname
• String lastname
• DateTime dob (date of birth)
• String street1
• String street2
• String city
• String state
• String zip
• String phone
• String email

}

In this embodiment, a particular consent record is an asset in the blockchain of channel 62 and includes at least a portion of the data shown in Table 2, below:

TABLE 2

asset Consent identified by consentid {

• String consentid
• String type
• DateTime grant
• DateTime expiry
• Boolean status
• String doc_type
• String doc_id
• String franchise
• String brand
• String program_name
• String tactic
• Strong channel

}

In this embodiment, a particular patient consent is an asset in the blockchain of channel 62 and includes the contents of Table 3, below:

TABLE 3

asset PatientConsent identified by PCid {

• String PCid [symbol]
Patient consentProvider [symbol]
Consents [ ] consents [symbol]
Grantee grantee

}

In this embodiment, a consent grantee is a network participant of channel 62 identified according to Table 4, below:

TABLE 4

participant Grantee identified by id {

• String id
• String name

}

Should a user make a request, made via application 22, to write a particular consent confirmation or other change to the blockchain, the chaincode for channel 62 is invoked on each peer P in channel 62 to trial the transaction through its respective blockchain copy and to provide an endorsement if the transaction would be acceptable to the peer P. In the event that the required endorsements provided back to the Fabric Interface 44 of the Node JS webserver 40 by peers P verify the endorsement policy for that chaincode has been satisfied, then the Fabric Interface 44 in turn interfaces with orderer 80 to provide an instruction to add the transaction to a next blockchain block. Pursuant to the instruction, orderer 80 orders the transactions into a new block and sends the new block to all organizations 64A, 64B, etc. in the channel to be added to respective copies of the blockchain maintained by peers P. As such, all peers P in the channel 62, even if from different organizations 64A, 64B etc., are meant to contain an accurate and up-to-date copy of the blockchain. Any other organizations with peers P in the channel 62 are similarly handled, and no other peers P or organizations that are not within channel 62 can access the blockchain of channel 62 either to read from it or write to it.

Various features are implemented using system 10, such as providing authorized users with the ability to search and view consents by different parameters, such as consent type, received date, expiry date referring to a date on which a consent, previously given, will expire, the individual's name, data of birth, ZIP or postal code, an identifier unique to the user or to the specific consent, and the consent status. Furthermore, system 10 enables an authorized user to update the status of a consent through its lifecycle from open, to pending approval, to approved, to expired, and so forth. System 10 also enables consents to be shared between and within enterprises according to chaincode and endorsement policies that correspond to proper and secure regulation of the respective consents.

In an embodiment, webserver subsystem 20 enables consent transactions to be manually loaded, batch loaded, or loaded using an automated mechanism from another system based on a scheduler. Consent data is stored in the blockchain, copies of which are maintained by each of the peers in a given channel to which the blockchain pertains.

In an embodiment, metadata corresponding to consents may be stored in the blockchain or otherwise securely stored, so that data supporting a consent or data proving a consent, such as an audio, video or image file including contents indicative of a patient's giving of the relevant consent, can be referred to for auditing or other regulatory purposes. For scalability, it may be preferred that such supporting documentation be stored separately, outside of the blockchain, from the consent data being stored within the blockchain.

In an embodiment, consents may be conveyed between entities in the system through appropriate authorizations, such as from a doctor to a subcontractor lab company doing lab work under the authorization of that doctor.

In an embodiment, various roles may be established for users accessing via the web application 22. For example, administrator users, read-only access, and the like. Furthermore, in embodiments, web application 22 deploys functionality enabling authorized users to add entities to the network, to add different kinds of consents and corresponding chaincodes and endorsement policies to the system, and to manage types of alerts (SMS, push notifications, emails) from within the system.

In an embodiment, both internal and external users of system 10 may be provided with the ability to be provided with information about the presence of a particular consent and/or to validate that a consent has been captured by system 10. Embodiments may provide alerts to designated users once a consent has changed status, such as when it has been captured, has been approved, or has expired, or has otherwise changed.

Embodiments log changes to consents throughout their lifecycles and provide user interface access to such logs and reporting tools so that consent lifecycles may be audited other otherwise explored.

FIG. 2 is a schematic diagram showing a hardware architecture for one or more components of the blockchain-based consent management system 10 of FIG. 1, according to an embodiment. As would be understood, components of blockchain-based consent management system 10 may be deployed using various load balancing schema, using cloud services such as Amazon Web Services (AWS), and the like, which themselves may deploy virtual servers to handle throughput on demand. Various implementations of the architecture using various techniques for load balancing, expansion, geographic locality, or the like, may be employed.

In an embodiment, computing system 1000 includes a bus 1010 or other communication mechanism for communicating information, and a processor 1018 coupled with the bus 1010 for processing the information. The computing system 1000 also includes a main memory 1004, such as a random access memory (RAM) or other dynamic storage device (e.g., dynamic RAM (DRAM), static RAM (SRAM), and synchronous DRAM (SDRAM)), coupled to the bus 1010 for storing information and instructions to be executed by processor 1018. In addition, the main memory 1004 may be used for storing temporary variables or other intermediate information during the execution of instructions by the processor 1018. Processor 1018 may include memory structures such as registers for storing such temporary variables or other intermediate information during execution of instructions. The computing system 1000 further includes a read only memory (ROM) 1006 or other static storage device (e.g., programmable ROM (PROM), erasable PROM (EPROM), and electrically erasable PROM (EEPROM)) coupled to the bus 1010 for storing static information and instructions for the processor 1018.

Computing system 1000 also includes a disk controller 1008 coupled to the bus 1010 to control one or more storage devices for storing information and instructions, such as a magnetic hard disk 1022 and/or a solid state drive (SSD) and/or a flash drive, and a removable media drive 1024 (e.g., solid state drive such as USB key or external hard drive, floppy disk drive, read-only compact disc drive, read/write compact disc drive, compact disc jukebox, tape drive, and removable magneto-optical drive). The storage devices may be added to the computing system 1000 using an appropriate device interface (e.g., Serial ATA (SATA), peripheral component interconnect (PCI), small computing system interface (SCSI), integrated device electronics (IDE), enhanced-IDE (E-IDE), direct memory access (DMA), ultra-DMA, as well as cloud-based device interfaces).

Computing system 1000 may also include special purpose logic devices (e.g., application specific integrated circuits (ASICs)) or configurable logic devices (e.g., simple programmable logic devices (SPLDs), complex programmable logic devices (CPLDs), and field programmable gate arrays (FPGAs)).

While not strictly required for hardware that does not interact directly with users, computing system 1000 may also include a display controller 1002 coupled to the bus 1010 to control a display 1012, such as an LED (light emitting diode) screen, organic LED (OLED) screen, liquid crystal display (LCD) screen or some other device suitable for displaying information to a computer user. In embodiments, display controller 1002 incorporates a dedicated graphics processing unit (GPU) for processing mainly graphics-intensive or other highly-parallel operations. Such operations may include rendering by applying texturing, shading and the like to wireframe objects including polygons such as spheres and cubes thereby to relieve processor 1018 of having to undertake such intensive operations at the expense of overall performance of computing system 1000. The GPU may incorporate dedicated graphics memory for storing data generated during its operations, and includes a frame buffer RAM memory for storing processing results as bitmaps to be used to activate pixels of display 1012. The GPU may be instructed to undertake various operations by applications running on computing system 1000 using a graphics-directed application programming interface (API) such as OpenGL, Direct3D and the like.

While not strictly required for hardware that does not interact directly with users, computing system 1000 may include input devices, such as a keyboard 1014 and a pointing device 1016, for interacting with a computer user and providing information to the processor 1018. The pointing device 1016, for example, may be a mouse, a trackball, or a pointing stick for communicating direction information and command selections to the processor 1018 and for controlling cursor movement on the display 1012. The computing system 1000 may employ a display device that is coupled with an input device, such as a touch screen. Other input devices may be employed, such as those that provide data to the computing system via wires or wirelessly, such as gesture detectors including infrared detectors, gyroscopes, accelerometers, radar/sonar and the like. A printer may provide printed listings of data stored and/or generated by the computing system 1000.

Computing system 1000 performs a portion or all of the processing steps discussed herein in response to the processor 1018 and/or GPU of display controller 1002 executing one or more sequences of one or more instructions contained in a memory, such as the main memory 1004. Such instructions may be read into the main memory 1004 from another processor readable medium, such as a hard disk 1022 or a removable media drive 1024. One or more processors in a multi-processing arrangement such as computing system 1000 having both a central processing unit and one or more graphics processing unit may also be employed to execute the sequences of instructions contained in main memory 1004 or in dedicated graphics memory of the GPU. In alternative embodiments, hard-wired circuitry may be used in place of or in combination with software instructions.

As stated above, computing system 1000 includes at least one processor readable medium or memory for holding instructions programmed according to the teachings of the invention and for containing data structures, tables, records, or other data described herein. Examples of processor readable media are solid state devices (SSD), flash-based drives, compact discs, hard disks, floppy disks, tape, magneto-optical disks, PROMs (EPROM, EEPROM, flash EPROM), DRAM, SRAM, SDRAM, or any other magnetic medium, compact discs (e.g., CD-ROM), or any other optical medium, punch cards, paper tape, or other physical medium with patterns of holes, a carrier wave (described below), or any other medium from which a computer can read.

Stored on any one or on a combination of processor readable media, is software for controlling the computing system 1000, for driving a device or devices to perform the functions discussed herein, and for enabling computing system 1000 to interact with a human user. Such software may include, but is not limited to, device drivers, operating systems, development tools, and applications software. Such processor readable media further includes the computer program product for performing all or a portion (if processing is distributed) of the processing performed discussed herein.

The computer code devices discussed herein may be any interpretable or executable code mechanism, including but not limited to scripts, interpretable programs, dynamic link libraries (DLLs), Java classes, and complete executable programs. Moreover, parts of the processing of the present invention may be distributed for better performance, reliability, and/or cost.

A processor readable medium providing instructions to a processor 1018 may take many forms, including but not limited to, non-volatile media, volatile media, and transmission media. Non-volatile media includes, for example, optical, magnetic disks, and magneto-optical disks, such as the hard disk 1022 or the removable media drive 1024. Volatile media includes dynamic memory, such as the main memory 1004. Transmission media includes coaxial cables, copper wire and fiber optics, including the wires that make up the bus 1010. Transmission media also may also take the form of acoustic or light waves, such as those generated during radio wave and infrared data communications using various communications protocols.

Various forms of processor readable media may be involved in carrying out one or more sequences of one or more instructions to processor 1018 for execution. For example, the instructions may initially be carried on a magnetic disk of a remote computer. The remote computer can load the instructions for implementing all or a portion of the present invention remotely into a dynamic memory and send the instructions over a wired or wireless connection using a modem. A modem local to the computing system 1000 may receive the data via wired Ethernet or wirelessly via Wi-Fi and place the data on the bus 1010. The bus 1010 carries the data to the main memory 1004, from which the processor 1018 retrieves and executes the instructions. The instructions received by the main memory 1004 may optionally be stored on storage device 1022 or 1024 either before or after execution by processor 1018.

Computing system 1000 also includes a communication interface 1020 coupled to the bus 1010. The communication interface 1020 provides a two-way data communication coupling to a network link that is connected to, for example, a local area network (LAN) 1500, or to another communications network 2000 such as the Internet. For example, the communication interface 1020 may be a network interface card to attach to any packet switched LAN. As another example, the communication interface 1020 may be an asymmetrical digital subscriber line (ADSL) card, an integrated services digital network (ISDN) card or a modem to provide a data communication connection to a corresponding type of communications line. Wireless links may also be implemented. In any such implementation, the communication interface 1020 sends and receives electrical, electromagnetic or optical signals that carry digital data streams representing various types of information.

The network link typically provides data communication through one or more networks to other data devices, including without limitation to enable the flow of electronic information. For example, the network link may provide a connection to another computer through a local network 1500 (e.g., a LAN) or through equipment operated by a service provider, which provides communication services through a communications network 2000. The local network 1500 and the communications network 2000 use, for example, electrical, electromagnetic, or optical signals that carry digital data streams, and the associated physical layer (e.g., CAT 5 cable, coaxial cable, optical fiber, etc.). The signals through the various networks and the signals on the network link and through the communication interface 1020, which carry the digital data to and from the computing system 1000, may be implemented in baseband signals, or carrier wave based signals. The baseband signals convey the digital data as unmodulated electrical pulses that are descriptive of a stream of digital data bits, where the term “bits” is to be construed broadly to mean symbol, where each symbol conveys at least one or more information bits. The digital data may also be used to modulate a carrier wave, such as with amplitude, phase and/or frequency shift keyed signals that are propagated over a conductive media, or transmitted as electromagnetic waves through a propagation medium. Thus, the digital data may be sent as unmodulated baseband data through a “wired” communication channel and/or sent within a predetermined frequency band, different than baseband, by modulating a carrier wave. The computing system 1000 can transmit and receive data, including program code, through the network(s) 1500 and 2000, the network link and the communication interface 1020. Moreover, the network link may provide a connection through a LAN 1500 to a mobile device 1300 such as a personal digital assistant (PDA) laptop computer, or cellular telephone.

Alternative configurations of computing systems may be used to implement the systems and processes described herein.

Electronic data stores implemented in the database described herein may be one or more of a table, an array, a database, a structured data file, an XML file, or some other functional data store, such as hard disk 1022 or removable media 1024.

Although embodiments have been described with reference to the drawings, those of skill in the art will appreciate that variations and modifications may be made without departing from the spirit, scope and purpose of the invention as defined by the appended claims.

For example, in an embodiment, a single entity may store all peer instances thereby centrally storing all copies of the blockchain. The peer instances may control access to respective blockchains, but they may be stored either physically or logically in a central manner rather than physically distributed as different machines.

In another embodiment, some of the peers are stored centrally and some are physically different machines.

In another embodiment, all peers are physically different machines. In an embodiment, transactions may be routed, based on access control, to the peers using an interface other than the application.

In an embodiment, organizations manage their own cryptographic blockchain identities, using them to sign transactions such as creating or updating consent before sending the transactions to the web application. This architecture may increase the certainty that data is being provided to system by an authorized party.

In an embodiment, the web application provides different levels of access control so that certain users within an organization can have modified or restricted access to data stored on the blockchain, according to the roles and responsibilities within the organization.

1. A blockchain-based consent management system comprising:
a Webserver subsystem configured to receive and handle authorized web user requests for access to and/or transactions corresponding to consent data for a blockchain, the Web server subsystem comprising a blockchain subsystem interface; and
a blockchain subsystem defining a channel having at least two organizations, corresponding chaincode and an endorsement policy, each of the at least two organizations having at least one peer, each of the at least one peer maintaining a blockchain copy, the blockchain subsystem comprising an orderer in communication with the blockchain subsystem interface.

2. The blockchain-based consent management system of claim 1, wherein the blockchain subsystem and the Webserver subsystem communicate such that authorized requests for transactions for the blockchain cause the Webserver subsystem to generate transaction proposals to be routed via the blockchain subsystem interface to the channel peers of the blockchain subsystem for individual endorsement, to receive endorsement responses from the peers, and to, in the event the endorsement responses collectively satisfy the endorsement policy, transmit the endorsed transactions to the orderer for inclusion in an additional block of the blockchain,
wherein the orderer is configured to cause the additional block to be stored in each of the peers' blockchain copy.

3. The blockchain-based consent management system of claim 1, wherein the Webserver subsystem is in communication with a certificate authority.

4. The blockchain-based consent management system of claim 1, wherein the Webserver subsystem comprises a first auto-scaling group in communication with a second auto-scaling group via a request queue, wherein the first auto-scaling group lodges requests corresponding to user requests in the request queue for consumption by the second auto-scaling group.

5. The blockchain-based consent management system of claim 3, wherein the first auto-scaling group comprises one or more Node JS webservers having user interface and application middleware components.

6. The blockchain-based consent management system of claim 3, wherein the second auto-scaling group comprises one or more Node JS webservers having instances of a hyperledger fabric interface.

7. The blockchain-based consent management system of claim 3, wherein the Webserver subsystem further comprises load balancing components for routing the user requests to Webserver instances in the first auto-scaling group.
